(19) United States
(12) Patent Application Publication
(10) Pub. No.: US 2012/0177609 A1
(43) Pub. Date: Jul. 12, 2012

(54) METHOD FOR USING LIBERATED DORMANT BACTERIOPHASE AND ENVIRONMENTAL STRESS TO REDUCE INFECTIOUS BACTERIA

(75) Inventors: DENISE MAILE RHODES, DEL MAR, CA (US); ALAN LEWIS GREENER, SAN DIEGO, CA (US)

(73) Assignee: INTERNALLE, INC., DEL MAR, CA (US)

(21) Appl. No.: 13/425,555

(22) Filed: Mar. 21, 2012

Related U.S. Application Data
(63) Continuation of application No. 12/088,951, filed on Jul. 28, 2008, now abandoned.
(60) Provisional application No. 60/724,003, filed on Oct. 5, 2005.

(51) Int. Cl. A61K 35/76; A01P 3/00; A01P 1/00; A01N 63/02; A61P 31/04

(52) U.S. Cl. 424/93.6

(57) ABSTRACT

A method for obtaining bacteriophages by stressing bacteria. The method involves isolating bacteria, propagating the bacteria, exposing the bacteria to at least one environmental stressing agent to induce excision of bacteriophage that are present in the bacterial genome. Multiple or individual environmental stressing agents may be applied. These liberated bacteriophages may be collected for purposes of treating pathogenic infections, protecting plants and agricultural products or general sterilization and sanitation.

### CROSS-REFERENCE TO RELATED APPLICATIONS

This application is a continuation of U.S. patent application Ser. No. 12/088,951, filed on Jul. 28, 2008, currently pending, which, in turn, is a 371 continuation of International application no. PCT/US06/38877, filed on Oct. 5, 2006, now expired, which, in turn, is a non-provisional of U.S. provisional application No. 60/724,003, filed on Oct. 5, 2005, now expired, the disclosures of which are hereby incorporated by reference in their entirety.

### BACKGROUND OF THE INVENTION

1. Field of the Invention

This invention relates to the excision and therapeutic application of bacteriophages for purposes of treating bacterial infections. The technique for excising lysogenized bacteriophages may also be useful for releasing lysogenized antimicrobial viruses from any microorganism for purposes of clinical treatment of infectious pathogens agricultural development and/or general antimicrobial sterilization affecting humans, animals, plants, and surfaces and aquatic environments.

2. Description of the Related Technology

Bacteriophages are a heterogeneous group of viruses that infect and replicate in bacteria and may exist in either a lytic or lysogenic form. In the lytic cycle, a bacteriophage lyses a host cell upon replication of the virion. In the lysogenic cycle, the viral genome of a bacteriophage is incorporated within the host DNA so that the virus is reproduced in the host's offspring. The virus remains dormant until the host cell begins to deteriorate, which induces activation of prophages. The prophages then initiate replication of the bacteriophage and the lytic cycle.

Bacteriophages were originally discovered and contemplated for antimicrobial applications in the early part of the 20^{th} century. The subsequent development of antibiotics for treating bacterial infections, however, diverted interest in pursuing therapeutic applications utilizing bacteriophages.

In recent years, numerous strains of emerging bacteria and microorganisms have developed resistance to conventional antibiotics. Some bacterial stains such as vancomycin resistant Enterococci and methicillin resistant Staphylococci have evolved resistance to all known antibiotics, endangering the public health. Therefore there exists a need to develop antimicrobial therapies to treat infections resistant to conventional antibiotics.

Because of the increase in antibiotic resistant bacteria, bacteriophage therapies, in use since the 1940s in certain European nations, are being re-considered. A number of patents, such as U.S. Pat. No. 6,896,882, teach the application of bacteriophages for treating infectious pathogens. Therapeutic pharmaceutical compositions incorporating bacteriophages may include an isolated bacteriophage specific to a bacterial host, a mixture of bacteriophages capable of infecting a bacterial host or a mixture of bacteriophages capable of infecting different bacterial hosts or different strains of the same bacterial host.

Typically, such therapies derive their bacteriophages by passively gathering or utilizing archived bacteriophages. A standard method for obtaining therapeutic bacteriophages involve isolating biomass surrounding bacteria which may have bacteriophages normally found in association with the bacteria. This biomass is then mixed with a nutrient broth and inoculated with a laboratory bacterial strain in order to find a susceptible host to amplify any bacteriophages within the broth. Upon isolation, the bacteriophages are tested for activity on a collection of laboratory hosts, such as VRE strains, to determine bactericidal efficacy. These methods have limited success because the collected bacteriophage may not be specific to the organism causing infection and/or because propagating the bacteriophage in a different bacterial host may not amplify the bacteriophage specific to the infection. The passive collection of bacteriophage surrounding the area of infection restricts the concentration and variety of available bacteriophages because only the bacteriophages which happen to be present at the moment of collection can be collected.

Other therapeutic treatments utilize archived bacteriophages to combat bacterial infections. These archived samples may not necessarily be specific to the bacterial strain at issue. Although the bacterial strain may be identified as originating from the same bacterial genus and species against which an archived bacteriophage has been previously shown effective, the bacteria is susceptible to genetic drift or mutation, which may render the bacterial strain not susceptible to the archived bacteriophage.

Bacteria that are found in nature are known to harbor lysogenic bacteriophage capable of becoming lytic by certain environmental stressing agents. This method was first discovered in the 1940s when E. coli K-12 was irradiated with ultraviolet light and the excision of the temperate bacteriophage lambda was observed. WIPO Publication no. 2002/040642 further discloses a method for reducing microbiological corrosion in processing plants and gas production facilities such as oil and gas wells, pipelines, and sewage plants by stressing a bacterium to activate dormant bacteriophages. The disclosed methods for stressing bacteria include exposure to UV light, heat, antibiotics, and chemicals toxic to the bacteria. The publication also discusses introducing bacteriophages into a bacterium prior to stressing.

Environmental stressing agents capable of eliciting the excision of these viruses or virus-like particles, however, are not completely understood and are not known for purposes of generating bacteriophage for treating pathogenic infections. Further, certain agents, i.e. ultraviolet light, may not be sufficient to release all integrated bacteriophages.

Accordingly, there is a need to provide improved methods for providing bacteriophage for use in various applications.

There is also a need to expand the repertoire of environmental stressing agents to include more rapid and effective ways of inducing lysogenic viruses and lysing microorganisms for various clinical and sterilization applications.

### SUMMARY OF THE INVENTION

This invention is directed to a novel method for treatment of bacteria with bacteriophages. In a first aspect of the invention, the method involves isolating at least one bacteria, propagating the bacteria, exposing the propagated bacteria to one or multiple environmental stressing agents to induce bacteriophages, collecting the bacteriophages, and administering the bacteriophages to a location with bacteria to cause a reduction the bacteria population.

In comparison to conventional therapeutic methods, therapeutic methods in accordance with the present invention involve active induction of the excision of bacteriophage by stressing bacteria. Active induction increases the probability that the generated bacteriophages are specific to the bacteria strain. Moreover, active induction releases bacteriophages which may not be available by passive collection of bacteriophages.

In a second aspect of the invention, bacteriophage is delivered to an infection site while simultaneously subjecting the infection site to environmental stress.

In a third aspect of the invention, environmental stress is applied to other organisms such as fungi to liberate viruses similar to bacteriophages.

### BRIEF DESCRIPTION OF THE DRAWINGS

FIG. 1 shows a table describing lysates from induced cells infecting various host cells and the sensitivity of similar host cells not obtained from induced cells, to the lysates.

FIGS. 2(a)-2(j) show the inoculation of 72 Ecor lysates on E. coli B, C and K12 and two Ecor strains.

### DETAILED DESCRIPTION OF THE PREFERRED EMBODIMENTS

The present invention is directed to certain novel methods for inducing bacteriophages or similar antimicrobial agents by stressing microorganisms such as bacteria, other prokaryotic organisms and fungi. While the inventors do not wish to be bound by a particular theory, it is believed that the underlying principal of this invention is that most or potentially all microorganisms are lysogens carrying DNA for dormant antimicrobial viral agents such as bacteriophages. For example, it is known that entire viral genomes or remnants of primordial viruses still persist in E. coli and other bacterial species. By stressing these microorganisms, it is possible to obtain microorganism specific antimicrobial agents from these microorganisms.

This invention is directed to a novel method for treatment of bacteria with bacteriophages. In a first aspect of the invention, the method involves isolating at least one bacteria, propagating the bacteria in various bacteria cultures, exposing the bacteria cultures to one or multiple environmental stressing agents to release dormant bacteriophages, collecting the bacteriophages after inducing stress, and administering the bacteriophages to a location with bacteria to cause a reduction the bacteria population. Preferably, the bacteriophages are administered topically or systemically to a site of infection. The bacteria may be selected from, for example, Mycobacteria, Staphylococci, Vibrio, Enterobacter, Enterococcus, Escherichia, Haemophilus, Neisseria, Pseudomonas, Shigella, Serratia, Salmonella, Streptococcus, Klebsiella and Yersinia.

The bacteria may be isolated, collected and propagated using traditional bacteriological protocols. Bacteria samples may be collected wherever the bacteria can be found, including from an infected patient, which may be a mammal, preferably, a human, or a non-patient source. For example, patient bacterial samples may be obtained from materials such as feces, urine, sputum and other bodily fluids. Non patient bacterial samples may include any contaminated surfaces, sewage and bodies of water. The bacteria is then grown in an appropriate media at various temperatures and incubated for varying durations suitable for the environmental stress to be employed.

The bacteria, preferably still in the cultures, are then subjected to different environmental stressing agents. Examples of environmental stressing agents include ultraviolet light, gamma irradiation, infrared irradiation, treatment with chemical mutagens (such as nitrous acid, hydroxylamine, ethyl methane sulfonate, mitomycin C, ethane methylsulfonate, nitrosoamine), hypertonic or hypotonic media, heavy metal additives, growth under high pressure, starvation by maintaining the bacterial culture in a stationary phase for a prolonged period of time, heat shock, cold shock, application of related or unrelated phage or other known methods in the field of bacterial genetics that cause perturbations in the bacterial growth cycle. These environmental stressing agents cause the release of dormant bacteriophage within the bacterial genome. The amount of ultra-violet light, heat, toxic or stress inducing chemicals or antibiotics to which the bacteria are exposed is predetermined in experiments to ensure that it stresses a bacterium but does not immediately kill it before producing bacteriophages which can then be used to kill other bacteria.

It is believed that the application of a stressing agent is capable of excising bacteriophages otherwise not available by passively isolating bacteriophages from collected sample since, at a given time of collection, not all potentially available bacteriophages will necessarily be present in the passively collected sample. The stressing agent appears to cause bacteria to release bacteriophages which may not be present in the passively collected sample. Once excised, the bacteriophages will replicate and lyse the host bacteria. Depending on the growth stage of the host, the released bacteriophages may infect other bacteria, replicate, and lyse the host bacteria.

Stressing agents may be applied individually or, in a preferred embodiment, a bacterial culture may be, simultaneously or over a period of time, exposed to multiple stressing agents. Application of different stressing agents releases bacteriophages in varying stages of development and bacteriophages that differ in identity or concentration. Subjecting a pathogenic bacterial population to multiple stressing agents simultaneously provides a more rapid and effective means for releasing lysogenized viruses.

After 1-24 hours of post-stress culturing, the bacteriophages may be lysed with chloroform, collected by filtration through a small 0.45 micron filter or isolated with cesium chloride density centrifugation and saved. Optionally, a further step of amplifying the bacteriophage, preferably in the same host as was used to induce the bacteriophage, may be employed.

In order to customize the therapeutic treatment, the collected bacteriophages may be specific to a bacterial host. When multiple stressors are used, cocktails of the various filtered bacteriophages may be used to treat a patient. This mixture of bacteriophages may be capable of infecting the same bacterial host. It is also possible for the bacteriophage mixture to infect different bacterial hosts or different strains of the same bacterial host. For added efficacy, the bacteriophages may be combined with other antimicrobial agents, such as antibiotics and chemotherapeutic agents. Table 1 lists examples of suitable additive antimicrobial agents and the corresponding bacterial infections which can be treated with the specified antimicrobial agents. The present invention, however, is not limited to the antimicrobial agents listed in Table 1.

TABLE 1

| | |
|---|---|
| E. coli (uncomplicated | trimethoprim-sulfamethoxazole |
| Urinary tract infection) | (abbrev. TMO-SMO), or ampicillin; |
| | 1st generation cephalosporins, |
| | Ciprofloxacin |
| E. coli systemic | ampicillin, or a 3rd generation |
| infection | cephalosporin; aminoglycosides, |
| | aztreonam, or a penicillin + a |
| | pencillinase inhibitor |
| Klebsiella pneumoniae | 1st generation cephalosporins; |
| | 3rd generation cephalosporins, cefotaxime, |
| | moxalactam, amikacin, chloramphenicol |
| Shigella (various) | ciprofloxacin; TMO-SMO, ampicillin, |
| | Chloramphenicol |
| Salmonella typhi | chloramphenicol; ampicillin or |
| | TMO-SMO |
| Salmonella non-typhi species | ampicillin; chloramphenicol, TMO-SMO, |
| | Ciprofloxacin |
| Yersinia pestis | streptomycin; tetracycline, |
| | Chloramphenicol |
| Enterobacter cloacae | 3rd generation cephalosporins, gentamicin, |
| | or tobramycin; carbenicillin, amikacin, |
| | aztreonam, imipenem |
| Haemophilus influenzae - | chloramphenicol or 3rd generation |
| meningitis | cephalosporins; ampicillin |
| Haemophilus influenzae - | ampicillin; TMO-SMO, cefaclor, |
| other H. influenza infections | cefuroxime, ciprofloxacin |
| Mycobacterium tuberculosis | isoniazid (INH) + rifampin or rifabutin, |
| and M. avium-intracellulare | the above given along with |
| | pyrazinamide +/or ethambutol |
| Neisseria meningitides | penicillin G; chloramphenicol, or |
| | a sulfonamide |
| Neisseria gonorrhoeae: | penicillin G; spectinomycin, ceftriaxone |
| penicillin-sensitive | |
| Neisseria gonorrhoeae: | ceftriaxone; spectinomycin, cefuroxime |
| penicillin-resistant | or cefoxitin, ciprofloxacin |
| Pseudomonas aeruginosa | tobramycin or gentamycin |
| | (+/−carbenicillin, aminoglycosides; |
| | amikacin, ceftazidime, aztreonam, |
| | Imipenem |
| Staphylococcus aureus: non- | penicillin G; 1st generation |
| penicillinase-producing | cephalosporins, vancomycin, imipenem, |
| | Erythromycin |
| Staphylococcus aureus: | a penicillinase-resisting penicillin; |
| penicillinase-producing | 1st generation cephalosporins, |
| | vancomycin, imipenem, erythromycin |
| Streptococcus pneumoniae | penicillin G; 1st generation |
| | cephalosporins, erythromycin, |
| | Chloramphenicol |

The bacteriophages may also be combined with any pharmaceutically acceptable carriers such as water, phosphate buffered saline solution, emulsions, wetting agents, propylene glycol, polyethylene glycol, vegetable oils, organic esters, alcoholic/aqueous solutions, sodium chloride, dextrose, fixed oils, fluid and nutrient replenishers and electrolyte replenishers.

An effective amount of a composition comprising one or more bacteriophages may be topically or systemically administered to a mammal, preferably to a human, to treat a microbial infection. The therapeutic compound may be formulated in any manner suitable for delivering the bacteriophage to the site of infection that does not inhibit its ability to infect and replicate within a host. In general, the pharmaceutical composition can be formulated as an injection, granule, tablet, pill, suppository, capsule, microbead, microsphere, liposome, suspension, salve, lotion, etc. Upon application, the number of infecting bacteria and hence the bacteria population will decline.

The effectiveness of bacteriophage therapy may depend on the sensitivity of the bacteria to the bacteriophages and concentration of the delivered bacteriophages. The instant method for excising bacteriophages using stressing agents significantly improves on therapeutic treatments of the prior art by increasing the probability and/or ensuring that the delivered bacteriophages are specific to the infecting organism. In comparison, standard methods utilizing archived bacteriophages or passively gathered samples from, e.g. a location surrounding a site of infection, which may contain bacteriophages, have limited success due to lack of bacterial sensitivity since the applied bacteriophages obtained via these methods may be derived from different or mutated bacterial strains. Additionally, these methods typically amplify the collected bacteriophages by inoculating them with various bacterial strains, which may contain dormant bacteriophages. Therefore, the host cells propagate a mixture of bacteriophages, many of which may not be specific to the bacteria of interest. The instant invention, avoids this problem by propagating bacterial host cells and stressing these hosts to obtain the relevant bacteriophages.

In a second embodiment, stressing bacteria at the site of infection, alone or in combination with administering bacteriophages, may be therapeutically beneficial for bacterial infections which are partially or completely resistant to antibiotics. Upon delivering the bacteriophages to an infection site, the area of infection may be simultaneously subjected to environmental stress. Concurrent application of bacteriophages and environmental stress serves two purposes: (1) since the stress is directly bactericidal, it lyses bacteria and (2) the bacteriophages, which are released upon exposure to environmental stress at the infection site, infect and lyse other bacterial host cells. This concurrent treatment may improve upon the amount and rate of reduction of pathogenic bacteria and may provide a reservoir of new bacteriophage to treat these antibiotic resistant organisms.

The newly isolated bacteriophages obtained by stressing an infection site, may also be archived and used in subsequent treatments for patients with acute infections. The specific mixture of bacteriophages obtained in this manner may be different than mixtures obtained using prior art methods. These bacteriophages may be applied directly to a patient or may be mutagenized and screened for more highly virulent pathogenic variants.

This dual treatment regimen involving stressing the infection site is particularly beneficial for acute and life threatening infections where little time may be available to isolate and propagate the infectious bacteria. By attacking the pathogenic host cell using two mechanisms capable of working independently or synergistically, it is possible to more effectively and quickly excise resident viruses from the pathogen genome, lyse the bacterial hosts and reduce the pathogenic bacteria.

Any of a variety of bacterial infections may be treated using bacteriophages according to the invention. The bacterial infection may be localized (i.e. contained within an organ, at a site of a surgical wound or other wound, within an abscess) or may be systemic (i.e. the subject is bacteremic, e.g., suffers from sepsis).

In a third embodiment, the method for excising viral antimicrobial agents such as bacteriophages may be applied to other microorganisms such as fungi for purposes of producing extracts capable of killing pathogenic organisms or capable of slowing or bringing to a halt the spread of the infection in patients including humans and other animals. Additionally, the method may be used to treat or protect plants, including horticultural or agricultural products, from harmful pathogenic organisms. It is also envisioned that the method would be useful for broad antimicrobial sterilization and sanitization applications. The antimicrobial agents may be used to treat any contaminated surface, including medical devices and food processing equipment, or any contaminated aqueous solution, including sewage, waste water or bodily fluids. For example, by applying environmental stress to a wall containing toxic mold, it may be possible to lyse existing mold spores.

### EXAMPLES

### Example 1

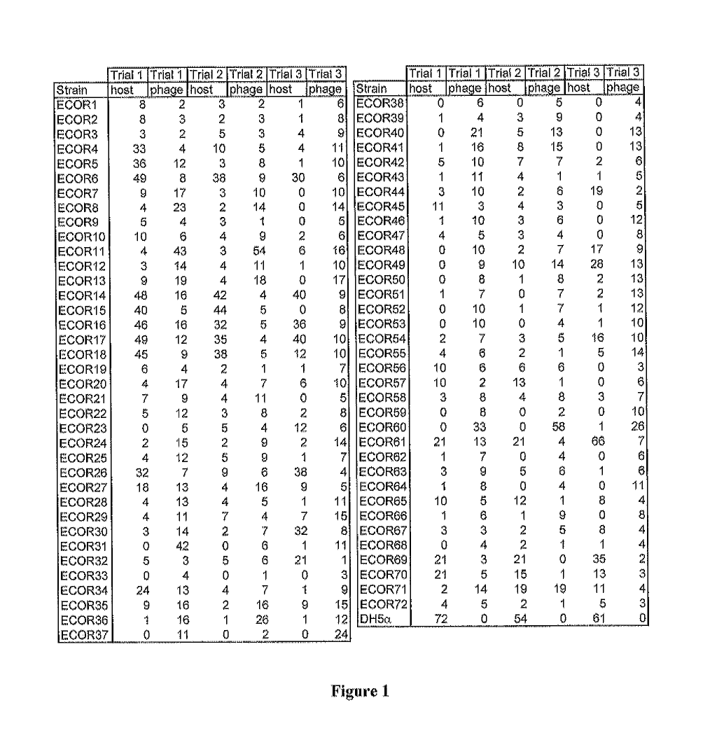

FIG. 1 presents experimental results demonstrating the excision of bacteriophages from E. coli after exposure to ultraviolet light. Host cells, ECOR-01-ECOR-72, were found to be susceptible to E. coli bacteriophage lysates and bacteriophage lysates derived from ECOR-01 to ECOR-72 (See Table 2) were found to be capable of infecting E. coli isolates.

Bacterial cultures were streaked from glycerol stocks onto 73 LB plates. Each plate contained 1 of the 73 bacterial strains listed in Table 2. Single colonies were inoculated into NZY broth and were grown shaking at 37° C. for about 18 to about 24. The cultures were used both as lawns for determining host cell sensitivity and irradiated with ultraviolet radiation for bacteriophage isolation. 1 ml of each of each culture was pelleted at 3000 g for 10 minutes. The culture was then washed with 1 ml of SM and pelleted. SM is a well known phage diluent. It is made with NaCl, MgSO4, and TrisCl.

After washing twice, the cell pellet was re-suspended in 800 ml of SM. The samples were then exposed to ultraviolet light at 777 microjoules/cm²×100. 200 μl of 5×NZY (5×NZY is NZY concentrated five times) and 50 mM CaCl₂ were added to each sample. The samples were then grown shaking for 18 hours. 100 μl of chloroform was added to each sample and vortexed. After the samples were pelleted at 4000 rpm for 15 min, lysate containing bacteriophage was removed. The step of adding 100 μl of chloroform may be repeated to remove any bacterial carryover.

3 mls of melted top agar medium was added to 300 μl of plating bacteria. Soft agar-bacterial suspension is poured onto the LB bottom agar to harden pursuant to the Adam's agar overlay method for routine phage production (Adams, M. H. Bacteriophages, Interscience Publishers, Inc., New York (1959)).

The isolation of induced bacteriophages was repeated three times. FIG. 1 shows the results for the three trial spottings. The host column shows how many different bacteriophage lysates derived from ECOR-01 to ECOR-72 were able to infect the bacterial host strain identified in each row. The phage column shows how many different bacterial strains were sensitive, capable of being infected by, the bacteriophage lysate derived from the strain identified in each row. These results demonstrate that lytic bacteriophage was released by the E. coli, the obtained bacteriophages can infect host cells of different strains and similarly a host cell may be susceptible to bacteriophages even those that were able to release phage.

TABLE 2
ECOR-01 A ON HN human (Female, 19 yr) USA (Iowa) healthy RM74A
ECOR-02 A ON H32 human (Male) USA (N.Y.), 1979 healthy STM1
ECOR-03 A O1 NM dog USA (Mass.) healthy W1R1(a)
ECOR-04 A ON HN human (Female, 5 yr) USA (Iowa) healthy RM39A
ECOR-05 A O79 NM human (Female, 56 yr) USA (Iowa) healthy
RM60A
ECOR-06 A ON NM human (Male, 8 yr) USA (Iowa) healthy RM66C
ECOR-07 A O85 HN orangutan USA (Wash.)* healthy RM73C
ECOR-08 A O86 NM human (Female, 20 yr) USA (Iowa) healthy RM77C
ECOR-09 A ON NM human (Female) Sweden healthy FN98
ECOR-10 A O6 H10 human (Female) Sweden healthy ANI
ECOR-11 A O6 H10 human (Female) Sweden UTI(C) C97
ECOR-12 A O7 H32 human (Female) Sweden healthy FN59
ECOR-13 A ON HN human (Female) Sweden healthy FN10
ECOR-14 A OM HN human (Female) Sweden UTI(P) P62
ECOR-15 A O25 NM human (Female) Sweden healthy FN3
ECOR-16 A ON H10 leopard USA (Wash.)* healthy RM191F
ECOR-17 A O106 NM pig Indonesia healthy RM200Q
ECOR-18 A O5 NM Celebese ape USA (Wash.)* healthy RM210F
ECOR-19 A O5 HN Celebese ape USA (Wash.)* healthy RM210J
ECOR-20 A O89 HN steer Bali healthy RM213I
ECOR-21 A O121 HN steer Bali healthy RM213K
ECOR-22 A ON HN steer Bali healthy RM215C
ECOR-23 A O86 H43 elephant USA (Wash.)* healthy RM183E
ECOR-24 A O15 NM human (Female) Sweden healthy FN33
ECOR-25 A ON HN dog USA (N.Y.) healthy MS1
ECOR-26 B1 O104 H21 infant USA (Mass.) healthy LL
ECOR-27 B1 O104 NM giraffe USA (Wash.)* healthy RM24J
ECOR-28 B1 O104 NM human (Female, 4 yr) USA (Iowa) healthy
RM52B
ECOR-29 B1 O150 H21 kangaroo rat USA (Nev.) healthy RM3A
ECOR-30 B1 O113 H21 bison Canada healthy RM10A
ECOR-31 E O79 H43 leopard USA (Wash.)* healthy RM12
ECOR-32 B1 O7 H21 giraffe USA (Wash.)* healthy RM28
ECOR-33 B1 07 H21 sheep USA (Calif.) healthy RM56C
ECOR-34 B1 O88 NM dog USA (Mass.) healthy WIR2(a)
ECOR-35 D O1 NM human (Female, 36 yr) USA (Iowa) healthy RM42B
ECOR-36 D O79 H25 human (Female, 20 yr) USA (Iowa) healthy
RM77B
ECOR-37 E ON HN marmoset USA (Wash.)* healthy RM44B
ECOR-38 D O7 NM human (Female, 21 yr) USA (Iowa) healthy RM75A
ECOR-39 D O7 NM human Sweden healthy FN104
ECOR-40 D O7 NM human Sweden UTI(P) P60
ECOR-41 D O7 NM human (Female, 22 yr) Tonga, 1982 healthy T44
ECOR-42 E ON H26 human (Male) USA (Mass.), 1979 healthy DAR1
ECOR-43 E ON HN human (Female) Sweden healthy FN36
ECOR-44 D ON HN cougar USA (Wash.)* healthy RM189I
ECOR-45 B1 ON HM pig Indonesia healthy RM201C
ECOR-46 D O1 H6 ape USA (Wash.)* healthy RM202F
ECOR-47 D OM H18 sheep New Guinea healthy RM211C
ECOR-48 D ON HM human (Female) Sweden UTI(C) C90
ECOR-49 D O2 NM human (Female) Sweden healthy FN90
ECOR-50 D O2 HN human (Female) Sweden UTI(P) P97
ECOR-51 B2 O25 HN infant USA (Mass.) healthy DD
ECOR-52 B2 O25 H1 orangutan USA (Wash.)* healthy RM73A
ECOR-53 B2 O4 HN human (Female, 4 yr) USA (Iowa) healthy RM33B
ECOR-54 B2 O25 H1 human USA (Iowa) healthy RM64A
ECOR-55 B2 O25 H1 human (Female) Sweden UTI(P) FN4
ECOR-56 B2 O6 H1 human (Female) Sweden healthy P106
ECOR-57 B2 ON NM gorilla USA (Wash.)* healthy RM71B
ECOR-58 B1 O112 H8 lion USA (Wash.)* healthy RM185S
ECOR-59 B2 O4 H40 human (Male) USA (Mass.), 1979 healthy SIL8
ECOR-60 B2 O4 HN human (Female) Sweden UTI(C) C89
ECOR-61 B2 O2 NM human (Female) Sweden healthy FN83
ECOR-62 B2 O2 NM human (Female) Sweden UTI(P) P69
ECOR-63 B2 ON NM human (Female) Sweden healthy FN21
ECOR-64 B2 O75 NM human (Female) Sweden UTI(C) C70
ECOR-65 B2 ON H10 Celebese ape USA (Wash.)* healthy RM202I
ECOR-66 B1 O4 H40 Celebese ape USA (Wash.)* healthy RM209I
ECOR-67 B1 O4 H43 goat Indonesia healthy RM217T
ECOR-68 B1 ON NM giraffe USA (Wash.)* healthy RM224H
ECOR-69 B1 ON NM Celebese ape USA (Wash.)* healthy RM45EM
ECOR-70 B1 O78 NM gorilla USA (Wash.)* healthy RM70B
ECOR-71 B1 O78 NM human Sweden (Female) ABU ABU84
ECOR-72 B1 O144 H8 human Sweden (Female) UTI(P) P68
DH5α ATCC ® Number 53868 Escherichia coli

These 72 E. coli strains are identified by the American Type Culture Collection (ATCC) as ECOR-01-ECOR-72. The Escherichia coli Reference Collection was established in 1984 by Howard Ochman and Robert K. Selander (J. Bacteriol. 157:690-693). The selection of these reference strains are representative of a collection of 2,600 E. coli isolates from natural populations.

### Example 2

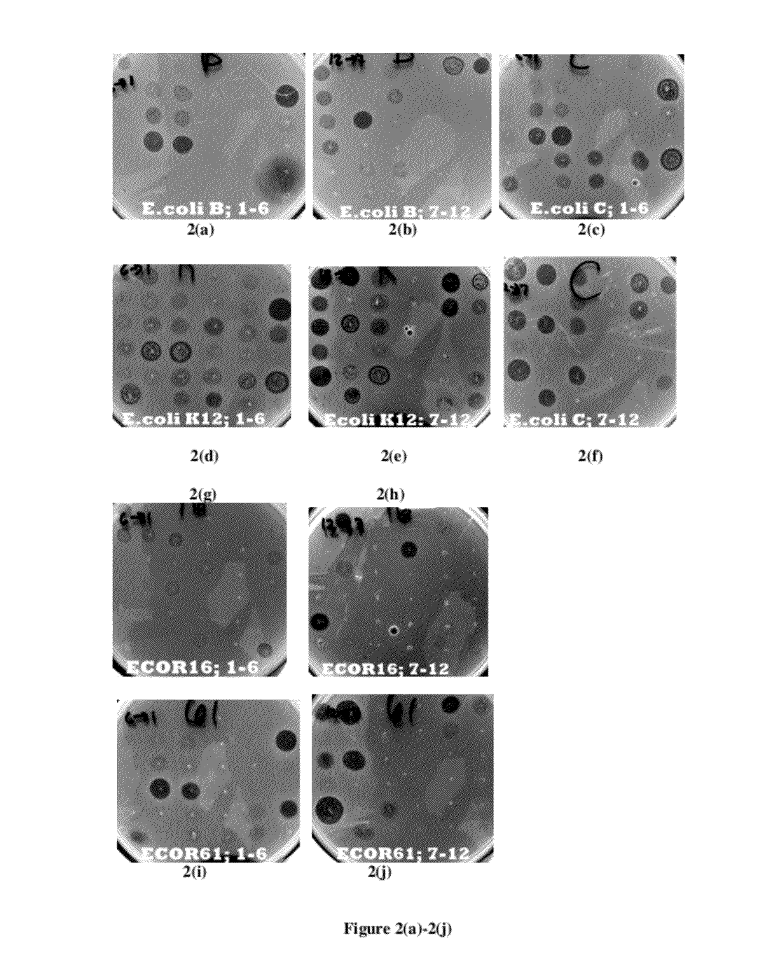

FIGS. 2(a)-2(j) show the inoculation of 72 Ecor lysates on E. coli B, C and K12 and two Ecor strains. The same method for inducing and preparing the bacteriophage as in Example 1 was employed. Three separate bacteriophage preparations of the 72 ECOR-1 to ECOR-72 were induced by stressing three of the same Ecor strains. The three lysates were combined in equal volumes. 3 μl of each lysate from Ecor 1-72 were then spotted on Ecor 16, Ecor 61, E. coli B strain BL21, E. coli C strain C2110 and E. coli K12 strain DH5. The bacteriophages were spotted on all five lawns in the same order. There are two plates of each bacterial strain, labeled 1-6 or 7-12 (See Tables 3 & 4), wherein each plate contains 36 of the 72 Ecor strain lysates on each lawn. The bacteriophages then lyse the cell lawn, releasing progeny bacteriophage, which can diffuse to and infect neighboring cells ultimately resulting in a circular area of cell lysis in a turbid lawn of cells.

TABLE 3
1-6

| 6 | 5 | 4 | 3 | 2 | 1 |
|---|---|---|---|---|---|
| 18 | 17 | 16 | 15 | 14 | 13 |
| 30 | 29 | 28 | 27 | 26 | 25 |
| 42 | 41 | 40 | 39 | 38 | 37 |
| 54 | 53 | 52 | 51 | 50 | 49 |
| 66 | 65 | 64 | 63 | 62 | 61 |

TABLE 4
7-12

| 12 | 11 | 10 | 9 | 8 | 7 |
|---|---|---|---|---|---|
| 24 | 23 | 22 | 21 | 20 | 19 |
| 36 | 35 | 34 | 33 | 32 | 31 |
| 48 | 47 | 46 | 45 | 44 | 43 |
| 60 | 59 | 58 | 57 | 56 | 55 |
| 72 | 71 | 70 | 69 | 68 | 67 |

The dark circles shown in FIG. 2(a)-2(j) represent lysis of the lawn by the bacteriophages. From the information in these figures, particularly that of E. coli K12, it is evident that stressing the bacteria cells makes it possible to excise a significant amount of bacteriophage therefrom. The clear zones are plaques formed in a lawn of cells due to lysis by phage.

While the invention has been described in detail and with reference to specific embodiments thereof, it will be apparent to one skilled in the art that various changes and modifications can be made therein without departing from the spirit and scope thereof.

1. A method for treating a bacterial infection comprising the steps of isolating at least one infecting bacteria from the bacterial infection, propagating the at least one infecting bacteria isolated from the bacterial infection, applying at least one environmental stressing agent to the propagated bacteria, obtaining bacteriophages from the at least one propagated infecting bacteria isolated from said bacterial infection, with the proviso that when the obtained bacteriophages are amplified, the obtained bacteriophages are amplified in host cells of a same strain as said at least one propagated infecting bacteria, and treating the bacterial infection with said obtained bacteriophages.

2. The method of claim 1, wherein the environmental stressing agent is selected from the group consisting of: phages, ultraviolet light, gamma irradiation, infrared irradiation, at least one chemical mutagen, hypertonic or hypotonic media, heavy metal additive, growth under high pressure, prolonged stationary phase retention, heat shock, cold shock and combinations thereof.

3. The method of claim 2, wherein the at least one chemical mutagen is selected from the group consisting of nitrous acid, hydroxylamine, ethyl methane sulfonate, mitomycin C, ethane methylsulfonate, nitrosoamine and mixtures thereof.

4. The method of claim 1, wherein the infecting bacteria is simultaneously or sequentially subjected to multiple environmental stressing agents.

5. The method of claim 1, wherein the bacterial infection is partially or completely antibiotic resistant.

6. The method of claim 1, wherein the obtained bacteriophages are not further amplified.

7. The method of claim 1, wherein the infecting bacteria are collected from a patient to be treated and at least some of the obtained bacteriophages are archived for future use.

8. The method of claim 1, wherein the infecting bacteria are collected from a non-patient source and at least some of the obtained bacteriophages are archived for future use.

9. The method of claim 1, wherein prior to treating the infection, the bacteriophages are combined with at least one antimicrobial agent selected from the group consisting of antibiotic agents and chemotherapeutic agents.

10. The method of claim 1, wherein the bacteriophages are prepared as a single natural isolate.

11. The method of claim 1, wherein the obtained bacteriophages comprise a mixture of at least two different bacteriophages.

12. The method of claim 1 further comprising the step of applying at least one environmental stressing agent at the site of bacterial infection to induce the excision and lytic development of endogenous bacteriophage.

13. The method of claim 12, wherein the at least one environmental stressing agent applied to the site of bacterial infection is selected from the group consisting of phages, ultraviolet light, gamma irradiation, infrared irradiation, at least one chemical mutagen, hypertonic or hypotonic media, heavy metal addition, growth under high pressure, prolonged stationary phase retention, heat shock, cold shock and mixtures thereof.

14. The method of claim 13, wherein the at least one chemical mutagen is selected from the group consisting of nitrous acid, hydroxylamine, ethyl methane sulfonate, mitomycin C, ethane methylsulfonate, nitrosoamine and mixtures thereof.

15. The method of claim 12, wherein the site of bacterial infection is simultaneously or sequentially subjected to multiple environmental stressing agents.

16. A method for excising and using bacteriophages or antimicrobial agents comprising the steps of isolating at least one microorganism from a surface, propagating the at least one microorganism isolated from the surface, subjecting the at least one propagated microorganism to at least one environmental stressing agent, obtaining lysogenic viral antimicrobial agents from said at least one propagated microorganism, with the proviso that when the obtained lysogenic viral antimicrobial agents are amplified, the obtained lysogenic viral antimicrobial agents are amplified in host cells of a same strain as said at least one propagated microorganism, and applying the lysogenic viral antimicrobial agents to a surface for the purpose of sterilizing or sanitizing the surface.

17. The method of claim 16, wherein the at least one environmental stressing agent is selected from the group consisting of phages, ultraviolet light, gamma irradiation, infrared irradiation, at least one chemical mutagen, hypertonic or hypotonic media, heavy metal addition, growth under high pressure, prolonged stationary phase retention, heat shock, cold shock and mixtures thereof.

18. The method of claim 17, wherein the at least one chemical mutagen is selected from the group consisting of nitrous acid, hydroxylamine, ethyl methane sulfonate, mitomycin C, ethane methylsulfonate, nitrosoamine and mixtures thereof.

19. A method for excising and using bacteriophages or antimicrobial agents comprising the steps of isolating at least one microorganism from a pathogenic infection of a plant or agricultural product, propagating the at least one isolated microorganism, subjecting the at least one propagated microorganism to at least one environmental stressing agent, and obtaining lysogenic viral antimicrobial agents from said at least one propagated microorganism, with the proviso that when the obtained lysogenic viral antimicrobial agents are amplified, the obtained lysogenic viral antimicrobial agents are amplified in host cells of a same strain as said at least one propagated microorganism, and applying the obtained viral antimicrobial agents to a plant or agricultural product for the purpose of protecting or treating the plant or agricultural product from pathogenic infection.

20. The method of claim 19, wherein the microorganism is selected from the group consisting of bacteria and fungus.

21. The method of claim 19, wherein the microorganism is not bacteria.

22. The method of claim 19, wherein the microorganism is bacteria and the antimicrobial agents are bacteriophages.

23. The method of claim 22, wherein the bacteria is of a genus selected from the group consisting of Mycobacteria, Staphylococci, Vibrio, Enterobacter, Enterococcus, Escherichia, Haemophilus, Neisseria, Pseudomonas, Shigella, Serratia, Salmonella, Streptococcus, Klebsiella and Yersinia.

24. The method of claim 19, wherein the microorganism is mold and the antimicrobial agents are used for purposes of sterilizing and sanitizing surfaces.

25. The method of claim 22, wherein the at least one environmental stressing agent is selected from the group consisting of phages, ultraviolet light, gamma irradiation, infrared irradiation, at least one chemical mutagen, hypertonic or hypotonic media, heavy metal addition, growth under high pressure, prolonged stationary phase retention, heat shock, cold shock and mixtures thereof.
